(19) United States
(12) Patent Application Publication
(10) Pub. No.: US 2023/0225716 A1
(43) Pub. Date: Jul. 20, 2023

(54) BIOPSY NEEDLE SET WITH DEFLECTION REDUCTION FEATURES FOR IMPROVED TARGETING

(71) Applicant: URO-1, Inc., Greensboro, NC (US)

(72) Inventors: Kevin Rackers, Greensboro, NC (US); Philip M. Allred, III, Kernersville, NC (US); Jack Snoke, Winston Salem, NC (US); Robert Deckman, San Bruno, CA (US); Ted Belleza, LaSelva Beach, NC (US); Bela Denes, Jacksonville, OR (US)

(21) Appl. No.: 18/119,042

(22) Filed: Mar. 8, 2023

Related U.S. Application Data
(63) Continuation-in-part of application No. 17/082,387, filed on Oct. 28, 2020.
Application No. 17/082,387 is a continuation-in-part of application No. 16/725,840, filed on Dec. 23, 2019, now abandoned.

(51) Int. Cl. A61B 10/02

(52) U.S. Cl. CPC A61B 10/0266; A61B 10/0241

(57) ABSTRACT

A biopsy needle set that includes an outer cannula and a core collector that slides therein and includes a cradle that has a trough-shaped open space formed between a keel and two rows of teeth rising from lateral ends of the keel. The cradle has an outer surface that conforms closely to the cannula's inside wall such that when the cradle partly extends distally from the cannula the outer surface of the cradle bears against the cannula's inside wall to thereby resist bending of the core collector's portion that extends distally from the cannula

### REFERENCE TO RELATED APPLICATION

This application is a continuation-in-part of, claims priority to, and incorporates by reference each of the following parent applications: (1) U.S. patent application Ser. No. 17/082,387 filed Oct. 28, 2020 and (2) U.S. patent application Ser. No. 16/725,840 filed Dec. 12, 2019.

### FIELD

This patent specification relates to medical devices for taking tissue samples including samples of prostate tissue. Some embodiments relate to a biopsy needle set comprising a core collector that remains radially centered relative to an outer cannula.

### BACKGROUND

When suspicious tissue is discovered in a patient's prostate or another region through manual examination or an imaging modality such as ultrasound, MRI, X-ray imaging or the like, it may be desirable to perform a biopsy procedure to remove one or more samples of that tissue to help determine if the tissue contains cancerous cells, other cells of interest, or to gain other information. A biopsy may be performed using an open or a percutaneous method. For prostate tissue, typically a core needle device is used that enters the prostate transrectally (TRUS) or transperineally (TPUS). There are complex prostate sampling devices that require two or more people for the procedure. And, there are simpler devices some of which can be disposed of after use on one patient. U.S. Pat. Nos. 5,546,957, 5,526,822, and 10,463,350, and published U.S. Patent application US 2016/0166331 A1 discuss examples of biopsy devices. The contents of said patents and published application are hereby incorporated by reference in this patent specification. A core biopsy device called Bard Magnum is believed to be commercially available from BD Bard in Tempe, Arizona and there are comparable devices available from other manufacturers.

An example of a prior art core biopsy set uses an outer cannula enclosing a biopsy needle with a notch. Initially, the biopsy needle is inside the cannula such that only the sharp tip of the biopsy needle protrudes distally. In this configuration, the cannula and biopsy needle as a unit are inserted in tissue until the distal end of the biopsy needle is at a desired position relative to a region of interest. Then, the biopsy needle shoots distally in the tissue, and some tissue enters the notch that is just behind (proximal from) the sharp tip. A fraction of a second later, the cannula shoots distally over the biopsy needle. The cannula has a sharp distal end that in this distal motion cuts off some of the tissue over the notch. Then the cannula and biopsy needle are withdrawn from the tissue as a unit, and the sample is taken out after moving the cannula axially back from the biopsy needle to expose the notch and the sample over it. Examples are discussed in U.S. Pat. Nos. 5,449,001, 10,188,422, and 10,478,159.

### SUMMARY OF THE DISCLOSURE

This patent specification describes a biopsy needle set comprising a cannula and a core collector that slidingly fits inside the cannula and has an axially extending cradle formed by a keel from the lateral sides of which two rows of teeth extend up. The teeth of each row are axially spaced from each other. In an axial view, the outer outline of the cradle extends over an arc of approximately half a circle that bears against the inside wall of the cannula to keep the cradle centered and resist bending a portion of the cradle that protrudes out of the cannula to collect a tissue sample. This configuration significantly improves placing the cradle at a desired position in living tissue and provides other significant benefits over known prior art biopsy needles that collect tissue samples in a notch that has no teeth.

According to some embodiments, a biopsy needle set comprises: an axially extending outer cannula that has an inside wall and a central axis; and an axially extending core collector that slidingly fits inside the outer cannula and has a sharp distal tip and an axially extending cradle proximal from the tip; wherein: the cradle comprises a trough formed between two axially extending rows of teeth rising from lateral ends of an axially extending keel; the cradle has an outer surface conforming to the cannula's inside wall; the keel has a cross-section that decreases in thickness toward the lateral ends thereof; the teeth of each row are axially spaced from each other; and the outer surface of the cradle is sized to bear against the cannula's inside wall and thereby resist bending of the cradle due to forces acting on the tip and cradle while and when a portion of the cradle is extending distally from the cannula.

According to some embodiments, the biopsy needle set further comprises one or more of the following: (a) some cross-sections of the cradle intersect only said keel and are at axially spaced portions of the cradle that lack said teeth; (b) some cross-sections of the cradle intersect only one of said teeth; (c) some cross-sections of the cradle intersect two of said teeth, one from each of said rows; (d) some cross-sections of the cradle intersect at least one of said teeth and some intersect none or said teeth; (e) said keel is sector-shaped in cross-section; (f) the outer surface of the cradle extends over an arc of approximately half a circle; (g) the cannula has a distal tip beveled along a first angle and the tip of the core collector is beveled along a second angle opposite the first angle; (h) at least some of the teeth have sharpened tips; (i) the surface of said keel facing the cannula's inside wall is a continuous curve in cross-section; (j) the surface of said keel facing said though is flat; and (k) the surface of said bearing against the inside wall of the cannula is continuous in cross-section.

According to some embodiments, a method of extracting a tissue sample comprises: providing a cannula that has an inner wall and a core that slidingly fits inside the cannula; wherein said core collector comprises a sharp distal tip and a cradle that is proximal from the tip and comprises an axially extending keel and two rows of teeth rising from lateral ends of the keel to thereby form a trough-shaped space between the keel and the teeth; forcing the core collector distally out of the cannula and into tissue from an initial position in which the cradle in inside the cannula such that the outer surface of the cradle bears against the cannula's inside wall as the cradle is moving out of the cannula and thereby resists bending of the cradle portion that has exited the cannula due to tissue exerting bending forces on the tip and cradle; and thereafter, forcing the cannula distally over the cradle to sever a sample of tissue that is between said trough shaped space and the cannula's inside wall.

According to some embodiments, the method further comprises one or more of the following: (a) the providing step comprises providing a core collector with a cradle some cross-sections of which intersect only said keel and are at axially spaced portions of the cradle that lack said teeth; (b) the providing step comprises providing a core collector with a cradle some cross-sections of which intersect only one of said teeth; (c) the providing step comprises providing a core collector with a cradle some cross-sections of which intersect two of said teeth, one from each of said rows; (e) the providing step comprises providing a core collector with a cradle some cross-sections of which intersect at least one of said teeth and some intersect none or said teeth; (f) the providing step comprises providing a core collector in which said keel is sector-shaped in cross-section; (g) the providing step comprises providing a core collector with a cradle the outer surface of which extends over an arc of approximately half a circle; and (h) the providing step comprises proving a core collector in which at least some of said teeth have sharpened tips.

### BRIEF DESCRIPTION OF THE DRAWINGS

FIG. 1 is a perspective view of a distal portion of a core collector, according to some embodiments.

FIG. 2 is a perspective view of a distal portion of another core collector, according to some embodiments.

FIG. 3A is a side elevation of a distal portion of a core collector, according to some embodiments.

FIG. 3B shows in a hatched area a cross-section of a core collector taken at the location indicated in FIG. 3A and further shows in the non-hatched areas core collector teeth that would be seen looking axially in the proximal direction from said cross-section (to the left in FIG. 3A), according to some embodiments.

FIG. 4 is a perspective view of distal portions of a prior art biopsy needle set comprising a biopsy needle in an outer cannula.

FIG. 5A is a cross-sectional view of a core collector like that of FIG. 3B and of an outer cannula surrounding the core collector, according to some embodiments.

FIG. 5B is a cross-sectional view like that of FIG. 5A but illustrating displacement of a core collector in an outer cannula, according to some embodiments.

FIG. 5C is a cross-sectional view through the notch of the prior art core collector of FIG. 4, illustrating displacement of the core collector in the outer cannula.

FIG. 6A is a side view of a core collector that partly extends distally from an outer cannula and illustrates forces acting on a tip of the core collector, according to some embodiments.

FIG. 6B is a side view of the prior art biopsy needle of FIG. 4, partly extending distally from an outer cannula, and forces acting at the tip of the biopsy needle.

FIG. 7A is a side view like FIG. 6A but showing downward deflection of a portion of the core collector that extends from the outer cannula, according to some embodiments.

FIG. 7B is a side view like FIG. 6B but showing downward deflection of a portion of a prior art biopsy needle that extends from the outer cannula.

FIG. 8A is a schematic illustration of a beam with a fixed connection to a wall, a force acting on the beam, and beam bending.

FIG. 8B is a schematic illustration of a beam twice as long as the beam of FIG. 8A, in a fixed connection to a wall but constrained in the middle from moving to the right.

FIGS. 9A and 9B are schematic illustrations of a cross-section of a rectangular beam and of an I-section beam, respectively.

FIG. 10A is a cross-sectional view of the prior art biopsy needle of FIG. 4 taken at the notch thereof, and FIG. 10B is a schematic cross-sectional view of a distal portion of a hypothetical core collector according to some embodiments, wherein a comparison of 10A and 10B shows a difference in vertical displacement of the two cross-sections due to forces acting at tips.

FIG. 11 is an enlarged perspective view of a distal portion of a core collector, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Further, reference numbers and designations that are alike in the various drawings indicate like elements. While terns such as over, under, lefthand and righthand have been used to describe geometric relationships, it should be clear that they refer to the illustrated orientations of elements and not to different orientations.

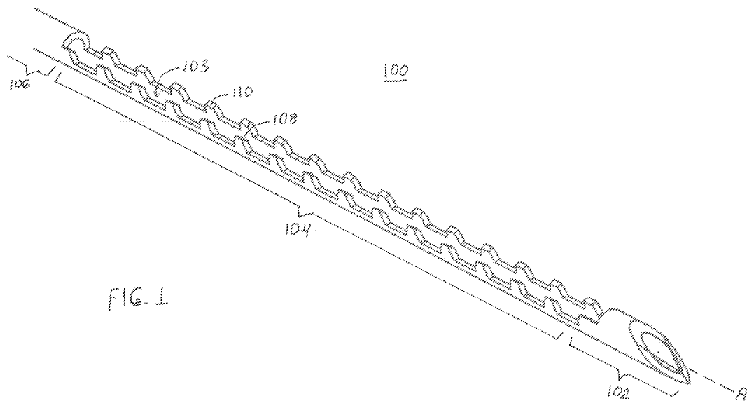

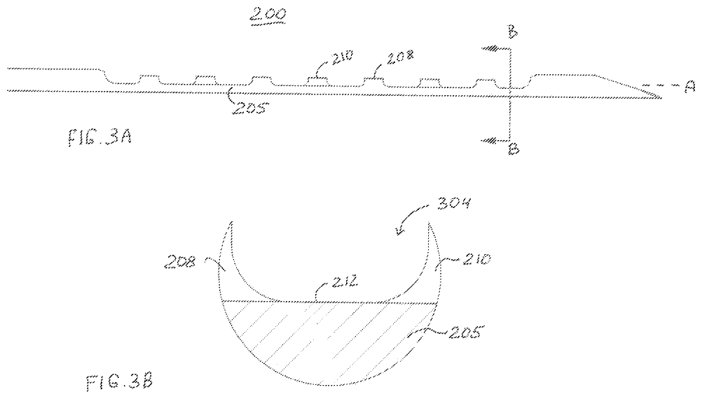

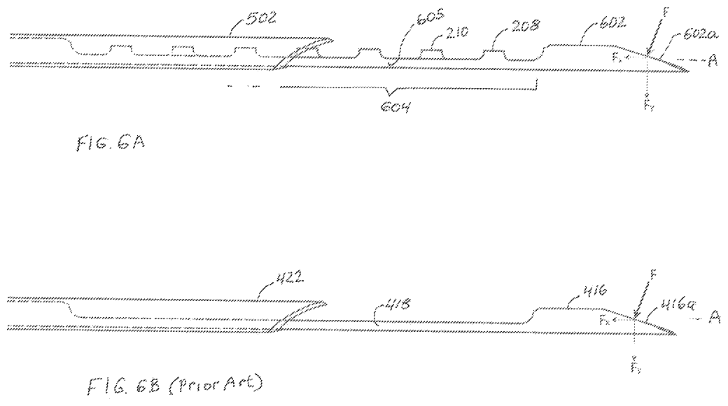

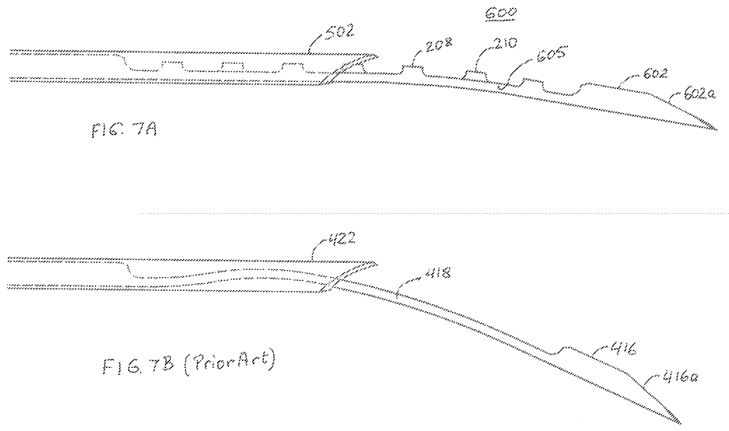

FIG. 1 is a perspective view of a distal portion of a core collector 100, according to some embodiments. Core collector 100 extends along a central axis A and includes a sharpened tip 102, a cradle 104 proximal from tip 102, and a tubular portion 106 proximal from cradle 104. Core collector 100 is configured to slidingly fit inside an outer cannula (such as cannula 502 in FIGS. 6A and 7A). Cradle 104 comprises a trough 103 formed between two axially extending rows of teeth 108 and 110 rising from lateral ends of a keel 105. Cradle 104 has an outer surface conforming to the cannula's inside wall, as further explained below in connection with FIGS. 6A and 7A. Keel 105 has a cross-section that decreases in thickness toward its lateral ends, as seen in FIG. 3B for example. Teeth 108 are axially spaced from each other along a lefthand row of teeth and teeth 110 are axially spaced from each other along a righthand row of teeth. The purpose and benefits of the keel and the rows of teeth are discussed further below.

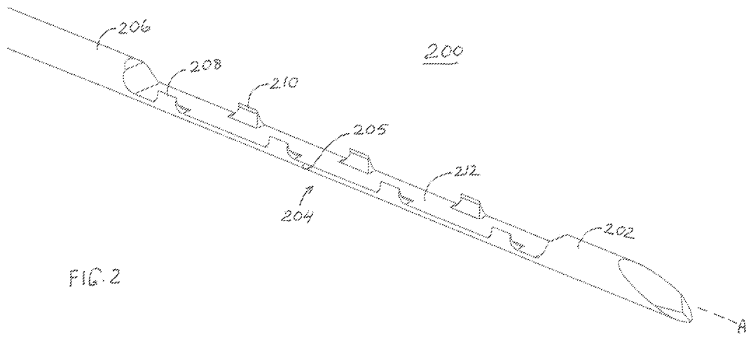

FIG. 2 is a perspective view of a distal portion of a core collector 200, according to some embodiments. Core collector 200 extends along central axis A and comprises a sharpened tip 202, a cradle 204 proximal from tip 202, and a tubular portion 206 proximal from cradle 204. Core collector 200 is configured to slidingly fit inside an outer cannula (such as cannula 502 in FIGS. 6A and 7A). Cradle 104 comprises a trough 203 formed between two axially extending rows of teeth 208 and 210 rising from lateral ends of a keel 205. Cradle 204 has an outer surface conforming to the cannula's inside wall, as further explained below in connection with FIGS. 6A and 7A. Keel 205 has an upper surface 212 and a cross-section that decreases in thickness toward its lateral ends, as seen in FIG. 3B for example. Teeth 208 are axially spaced from each other along a lefthand row of teeth and teeth 210 are axially spaced from each other along a righthand row of teeth. The purpose and benefits of the keel and the rows of teeth are discussed further below.

FIG. 3A is a side elevation of a distal portion of core collector 200 that includes a lefthand row of teeth 208 and a righthand row of teeth 210.

FIG. 3B shows a hatched area representing a cross-section of keel 205 taken at plane B-B indicated in FIG. 3A. Plane B-B intersects an axially extending portion of cradle 204 in which there are no teeth 208 or 210. The non-hatched areas 208 and 210 show teeth that would be seen looking axially in the proximal direction (to the left in FIG. 3A, as indicated by arrows)—a tooth 208 of the lefthand row of teeth and a tooth 210 of the righthand row of teeth, according to some embodiments. FIG. 3B also shows upper surface 212 of keel 205 and a trough 304 formed by upper surface 212 and teeth 208 and 210.

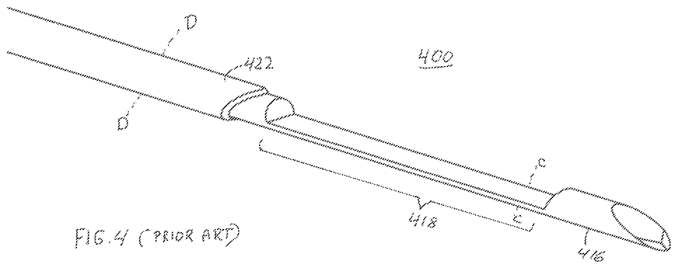

FIG. 4 is a perspective view of distal portions of a prior art biopsy needle set 400 comprising a biopsy needle 414 that has a sharp distal tip 416, a notch 418, and a solid rod 420. A part of solid rod 420 and all of notch 418 and tip 416 protrude distally from a distal end of an outer cannula 422 that has a sharp distal end. In prior art practice, needle set 400 has an initial position (not shown) in which biopsy needle 414 is retracted in outer cannula 422 such that only tip 416 protrudes, and in that initial position is inserted in a patient body such that tip 416 is at or near anatomy from which to take a tissue sample. Biopsy needle 414 then shoots forward from cannula 422 to thereby place notch 418 in or under the tissue to be sampled. A very short time later, cannula 422 shoots forward over biopsy needle 418 to sever and surround tissue that is immediately above notch 418. The prior art example of FIG. 4 differs from the examples illustrated in FIGS. 1, 2, 3A and 3B in that it lacks a cradle in a core collector and teeth. Differences in performance are discussed further below.

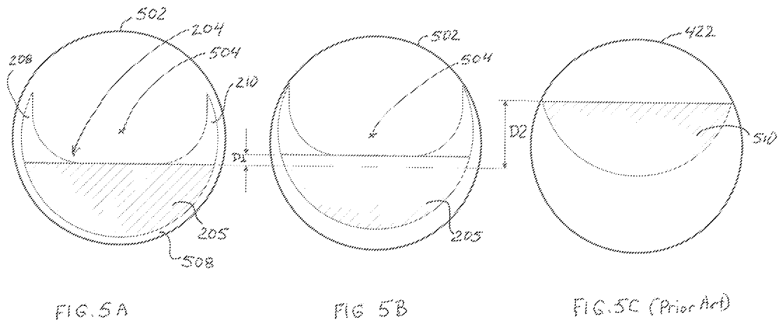

FIG. 5A shows a view of core collector 200 that is like the view of FIG. 3B but in addition shows schematically a cross-section of outer cannula 502 that has a geometric center 504 coinciding with central axis A of FIG. 2. As seen in FIG. 5A, cradle 204 of core collector 200 is illustrated as comprising a hatched portion representing a cross-section of keel 205 and non-hatched portions representing teeth 208 and 210. Cradle 204 is radially centered relative to geometric center 504 in that the outer surface of cradle 204, i.e., the outer surfaces of keel 205 and teeth 208 and 210, are against the inside wall of cannula 502, with just enough space 508 to allow for relative axial motion between cannula 502 and cradle 204 of core collector 200.

FIG. 5B is otherwise like FIG. 5A but shows a vertical displacement, over a distance D1, of cradle 204 of core collector 200 relative to cannula 502. For illustrative purposes the vertical displacement is exaggerated and can represent more displacement than in an actual needle set of a cannula and a cradle of a core collector such as core collector 200 or core collector 100. Vertical displacement is limited by the outside surfaces of keel 205 and of the tips of teeth 208 and 210 bearing against the inside wall of cannula 502.

FIG. 5C shows, for comparison, a cross-section 510 of notch 418 of prior art biopsy needle 400 (FIG. 4) taken at plane C-C and surrounded by a cross-section of outer cannula 422 (FIG. 4) taken at plane D-D. This relationship between notch 418 and cannula 422 can occur while notch 418 is advanced distally from cannula 422. As seen in FIG. 5C, the fit between notch 510 and cannula 422 can allow much greater vertical displacement, over a vertical distance D2 greater than D1.

Comparing FIG. 5B with FIG. 5C, the space in cradle 204 for a tissue sample has a much greater cross-sectional area than space 512 in the prior art example, for same diameter outer cannula.

FIG. 6A is a side view of a core collector 600 that partly extends distally from outer cannula 502. Core collector 600 can be like core collector 100 of FIG. 1 or core collector 200 of FIG. 2 and has a cradle 604 between a lefthand row of teeth 608 and a righthand row of teeth 610 and an upper surface such 612 of keel 605 that is like surface 212 in FIG. 2. Tip 602 ends distally and ends in a sharp bevel 602a. Schematically illustrated is a force F that acts on bevel 602a while core collector 602 moves distally through tissue. Force F can be decomposed in a horizontal component Fx and a vertical component Fy. Component Fx is due mainly to tissue resisting distal movement of core collector 602 and component Fy is mainly due to tissue pushing down as core collector 600 shoots distally through tissue, because of the inclined plane of bevel 602a.

FIG. 6B is a side view of prior art biopsy needle 414 of FIG. 5, partly extending distally from outer cannula 422, and a force F acting on bevel 416a of tip 416, decomposed into components Fx and Fy.

FIGS. 7A and 7B schematically illustrate differences in bending between core collector 600 of FIG. 6A and prior art biopsy needle 400 of FIG. 6B, under comparable conditions of partial extension from cannulas during insertion into tissue, assuming same diameter outer cannulas 502 and 422, same amount of force F, and same materials. As illustrated, the portion of cradle 604 that extends from cannula 502 bends down significantly less than the portion of notch 418 of biopsy needle 400 that extends the same distance out of cannula 422. This resistance to bending of cradle 604 is due mainly to the portions of keel 605 and teeth 208 and 210 that still are inside cannula 502 bearing against the inside wall of cannula 502, in the manner illustrated in FIGS. 5A and 5B and may be partly due to a stiffening effect of the teeth.

The resistance to bending is particularly significant when force F is greater. Force F increases with an increase in density of tissue being penetrated by core collector 600 and with the presence of denser nodules in the path of tip 602. Greater bending, such as seen in FIG. 7B, can have a number of undesirable consequences including failure to collect a sample from an intended location in tissue, collecting a lower volume of tissue than desired, and distorting the tissue sample in notch 418 as cannula 422 shoots distally over notch 418 and at a later time, when cannula 422 is pulled back from biopsy needle 400 to expose the tissue sample in notch 418 for retrieval. Note that as illustrated in FIG. 7B, the portion of notch 418 of biopsy needle 400 that still is inside cannula 422 also bends, as it is not vertically constrained in cannula 422.

In contrast, the configuration illustrated in FIG. 7A, which keeps cradle 604 much better centered relative to central axis A while in cannula 502, can significantly increase the likelihood of placing cradle 604 at an intended position in tissue, of collecting a greater volume of tissue, and of maintaining structural integrity of a tissue sample collected as cannula 502 shoots distally over cradle 604. Teeth 208 and 210 additionally help is holding tissue in place relative to cradle 604 while core collector 600 is shooting distally in tissue, while canula 502 is shooting distally over core collector 600, and while cannula 502 is being pulled back, proximally, from core collector 600 to expose a tissue sample for retrieval.

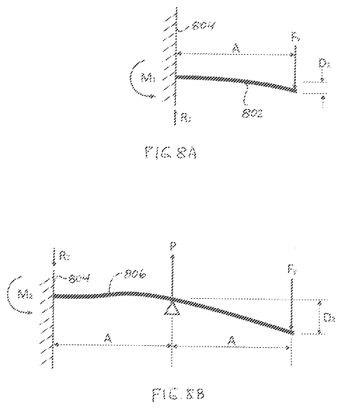

FIGS. 8A and 8B schematically illustrate bending of beams constrained differently and are intended to help in discussing physics that may be involved in some of the actions of core collectors such as 100, 200, and 600 when used for biopsy, although the actual action of such core collectors is much more complex, and some aspects of such action may not be entirely understood or explainable using such physics principles. FIG. 8A is a schematic illustration of a beam 802 with a fixed connection to a wall 804 and a force Fy acting on the beam at its distal end, and FIG. 8B is a schematic illustration of a beam 806 that is otherwise like beam 802 and has the same force Fy acting on its distal end but is twice as long, is in a fixed connection to a wall 808 and is constrained in the middle 810 from moving to the right

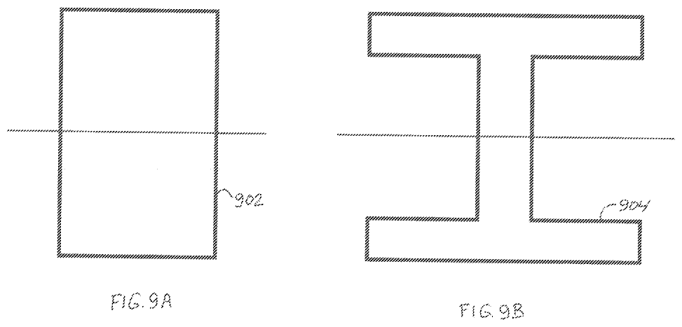

FIGS. 9A and 9B are schematic illustrations of a cross-section of a rectangular section beam 902 and of an I-section beam 904 that has the same cross-sectional area, respectively, and are discussed further below in connection with beam bending principles.

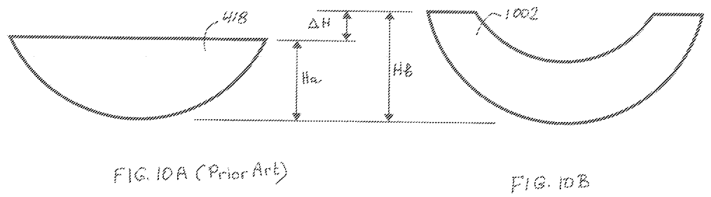

FIG. 10A is a cross-sectional view of prior art notch 418 of FIG. 4 and, for comparison, FIG. 10B is a schematic a cradle viewed in an axial direction from a cross-section. These figures may help in the discussion of beam bending further below.

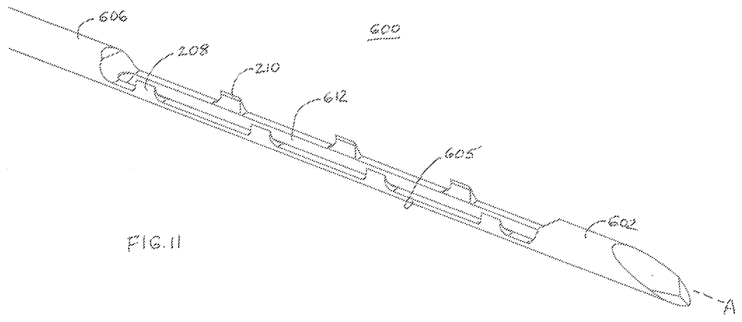

FIG. 11 is an enlarged perspective view of a portion of a cradle of a core collector such as core collectors 100, 200 and 600. If for core collector 600, FIG. 11 shows keel 605 extending along central axis A, upper surface 612 of keel 605, teeth 208 and 210, tubular portion 606 of the core collector, and tip 602.

In typical use, a needle set such as in FIG. 6A has an initial relative position of cannula 502 and core collector 600 in which cradle 604 is substantially inside cannula 502 and tip 602 protrudes from the cannula. While in this initial position, cannula 502 with core collector inside the cannula are forced into tissue until tip 602 is at a desired position relative to a lesion or other volume of tissue from which a tissue sample should be taken. Core collector 600 then shoots distally from cannula 502, typically driven by a spring, such that cradle 604 comes to rest in or under tissue to be sampled. Tissue then springs back and naturally enters trough 304 (FIGS. 3B, 5A and 5B), and a very shot time later cannula 502 shoots distally over cradle 604 and cuts a tissue sample that is in the space between cradle 604 and the inside wall of cannula 502. Teeth 208 and 210 help hold tissue that the teeth have engaged and resist axial displacement of such tissue relative to the teeth and thus help keep the tissue sample from axial and other distortion and help maintain its volume and integrity. In addition, when later the cannula and core collector are withdrawn as a unit from the tissue the teeth help keep the tissue sample in place relative to the cradle and help maintain the sample's volume and structural integrity.

The configuration of keel and teeth discussed above helps significantly with the important function to reduce the amount of deflection of the core collector during collection of a tissue sample. The reduced deflection action is important both while the core collector is being forced distally from the cannula into tissue and when the cannula shoots distally over the core collectors to cut a tissue sample from surrounding tissue. This configuration also helps when the tissue sample is between the cradle and the cannula and the cannula is pulled back from the core collector to be thereby exposed for retrieval.

Minimizing cradle deflection is important because biopsies are often collected in areas close to other organs or internal features that can be damaged if cut or punctured. A needle that deflects more than a few millimeters when collecting tissue from a tumor that is only a millimeter or two away from an organ like the bladder or the urethra in a prostate can cause major internal damage.

A core collector such as 100, 200, and 600 can help reduce cradle deflection in at least two ways: (1) by reducing core collector tip deflection by better constraining a cradle prior to the cradle shot distally out of the cannula, and (2) by providing increased strength to a cradle when protruding distally from the cannula by using a geometry that has an improved bend resistance without reducing the collection area of the cradle for a tissue sample.

If force Fy shown in FIGS. 6A and 6B as acting on tip 602 or 416 is sufficiently small and cradle 604 or notch 418 is sufficiently strong, cradle 604 or notch 418 may not deflect much and core collector 604 or biopsy needle 414 may travel in a straight line along central axis A. This may be the case if core collector 600 or biopsy needle 414 is fired into very soft tissue or in a medium such air or liquid. However, if core collector 600 or biopsy needle 414 is fired into a tissue which is sufficiently dense, such as prostate tissue, force F can increase to the point where the Fy component is large enough to cause cradle 604 to bend or notch 418 to bend more. With a large enough Fy component, cradle 604 can bend as illustrated in FIG. 7A and prior art notch 418 can bend much more as illustrated in FIG. 7B.

Regarding physics involved in beam bending, consider the common example of a cantilever beam's reaction to a force applied at the end of the beam. FIG. 8A shows a beam with a fixed connection at a wall. A fixed connection is one that does not allow rotation or translation. When a force F_{y} is applied to the beam, the magnitude of deflection can be estimated based on the following equation:

$${D}_{1} = \frac{{F}_{y} * {A}^{3}}{3 * E * I} = \frac{{F}_{y} * {A}^{3}}{E * I} ( 0.333 )$$

where:
D₁=Deflection at end of beam
F_{y}=Force at end of beam
A=Length of beam
E=Modulus of Elasticity
I=Rectangular Moment of Inertia
R₁=Reaction force at the wall
M₁=Reaction Moment at the wall

The reaction force R₁ and moment M₁ at the wall are the result of the force applied to the end of the beam. See Mark's Standard Handbook for Mechanical Engineers, 9th Edition, Section 5, Mechanics of Materials.

Another beam loading condition is shown in FIG. 8B. In this scenario there is a beam with a fixed constraint at a wall, a simple support constraint in the center of the beam and a force F_{y} applied to the end of the beam. A simple support constraint is one that does not allow translation but does allow rotation. In addition to the notation given above for FIG. 8A, the case of FIG. 8B has the following notations:
D₂=Deflection at end of beam
P=Force due to simple support constraint
R₂=Reaction force at the wall
M₂=Reaction Moment at the wall

The deflection for the beam in the FIG. 8B scenario can be determined with the following common equations (See Handbook referenced above):

$${F}_{y} = \frac{5}{16} P \\ {D}_{2} = \frac{7 P * {( 2 A )}^{3}}{768 * E * I}$$

Combining the two above equations and simplifying produces the following equation:

$${D}_{2} = \frac{{F}_{y} * {A}^{3}}{E * I} ( 0.467 )$$

In principle, if we assume the beams in FIG. 8A and FIG. 8B have the same cross-section geometry and are made of the same material, then their Rectangular Moment of Inertia, I, and Modulus of Elasticity, E, are the same. If we also assume that the length of beam in FIG. 8B is twice the length of the beam in FIG. 8A, then the dimension A is the same length in both scenarios. With these assumptions, we can compare deflections when both scenarios experience the same downward F_{y} force. The ratio of D₁ and D₂ can be determined as follows based on the above equations:

$$\frac{D 1}{D 2} = \frac{\frac{{F}_{y} * {A}^{3}}{E * I} ( 0.333 )}{\frac{{F}_{y} * {A}^{3}}{E * I} ( 0.467 )} = \frac{0.333}{0.467} = 0.71$$

The equation can also be given as D₁=0.71*D₂. Stated another way, it can be said that the beam in FIG. 8A deflects 29% less than the beam in FIG. 8B.

It can be observed in FIGS. 7A and 7B that the cradle in FIG. 7A is constrained in a way that has some similarities to the way the beam in FIG. 8A is fixed to the wall constraint. This is because cannula 502 keeps the portion of cradle 604 that still is inside cannula 502 from translating upward and also provides a moment constraint that resists rotation cradle 604 at the point it exits cannula 502. This reaction moment on cradle 604 is in some ways similar to the Mi moment on the beam. Prior art biopsy needle notch 418 of FIG. 7B is not constrained against rotation at the point it exits cannula 428. The constraint at the end of cannula 422 is in some ways similar to a simple constraint shown in the center of the beam in FIG. 8B. The main rotation constraint is only provided where notch 418 transitions to the full diameter of the biopsy needle shaft.

The portions of cradle 604 and of notch 418 that still are inside cannulas 502 and 422, respectively, are represented by the dashed lines. The bending up of notch 418 inside cannula 422 is greater than any bending of the portion of cradle 604 that still is inside cannula 502 because notch 418 can drift up inside cannula 422 as illustrated in FIGS. 5C and 7B. In the comparative example of FIGS. 7A and 7B, cradle 604 has the same length as notch 418, and keel 605 has the same thickness as notch 418. Both cradle 604 and notch 418 are examined at the point where they are half-way extended beyond the respective cannulas 502 and 422.

One difference between the examples of FIG. 7A and 7B and those of FIGS. 8A and 8B is that cradle 604 does not have a complete fixed constraint. There is some clearance between cradle 604 and cannula 502, which typically is very small, just to allow cradle 604 to slide within cannula 502 without binding. As illustrated in an exaggerated manner in FIG. 5B, this small clearance can allow for some upward translation of the cradle 604 within cannula 502.

Another difference from the beam examples is based on a static load condition and a single point of analysis. If the simple constraint in FIG. 8B were placed at a location other than the middle of the beam, the deflections would be different. In contrast, since the force Fy acting on tip 602 of core collector 600 is generated by motion through tissue, analysis of the loads at a particular time splice can help appreciate basic dynamics but cannot predict or explain the compete behavior of core collector 600 it tissue sampling. For instance, in using prior art biopsy needle 400 and notch 418 first starts its travel out of cannula 422, a static analysis could show little difference from the behavior of core collector 600. However, just beyond the middle of its travel, a static analysis would show a bigger deflection difference.

An additional difference to consider is that the downward force the tissue exerts on tip 602 or 416 is increased as the angle between the sloped surface 602a and 416a is increased. This means that if cradle 604 or notch 418 starts to dive downward, the force causing it to dive downward increases, which in turn causes it to dive even more. The example analysis looked at the half-way point in the travel out of the cannula and assumed that the deflecting force was the same for cradle 604 and notch 418; however, if notch 418 had already started deflecting before the half-way point, then the downward force on tip 416 would be larger than that on tip 602. The result of this phenomena would tend toward causing a greater deflection of notch 418 than of cradle 604.

The beam deflection equation shown above has a term “I” that is called the Rectangular Moment of Inertia. This is a geometric term that is directly related to the bending resistance a structure provides based on its cross-section and the distribution of the cross-section's area about a specific axis or centroid. A more in-depth discussion of the Moment of Inertia can be found in Mark's Standard Handbook for Mechanical Engineers, 9th Edition, Section 5, Mechanics of Materials. To help facilitate discussion of the Moment of Inertia as it relates to biopsy, consider the two areas shown in FIGS. 9A and 9B. Each image represents the cross section of a different shape beam. FIG. 9A represents a beam with a purely rectangular cross-section while FIG. 9B represents a beam with a cross-section that is typically referred to as an I-Beam cross-section.

Assume that the area of the cross section in FIG. 9A is equal to the area of the cross-section in FIG. 9B. Further assume that Axis Xa in FIG. 9A and Axis Xb in FIG. 9B bisect the cross sections so there is an equivalent area above and below each of these axes. Based on the Mark's Standard Handbook, the moment of inertia for the rectangular beam in FIG. 9A about the central axis Xa can be estimated and represented as Ia. Likewise, the moment of inertia for the I-Beam in FIG. 9B can be estimated about the central axis Xb and represented as lb. The moment of inertia for the rectangular beam, Ia, will be less than the moment of inertia for the I-Beam, Ib. This phenomenon is directly related to how the I-Beam has a greater percentage of its area distributed at a larger distance from the central axis than the area of the rectangular beam. From the beam bending equation shown above it can be observed that the deflection of a beam is inversely proportional to the moment of inertia. So, a larger moment of inertia will help reduce the amount of deflection of a beam. For the example of a rectangular beam vs an I-Beam, it can be concluded that if both beams were subjected to the same load conditions as shown in FIGS. 8A and 8B and both beams were made of the same material with the same Modulus of Elasticity, then the deflection of the I-Beam would be less than the deflection of the rectangular beam.

FIG. 10A shows the cross section of prior art notch 418. The height from the bottom of notch 418 to its top surface is Ha. FIG. 10B illustrates a cross-section 1002 of a hypothetical cradle of a core collector that is cup-shaped and has the same area as the cross-section in FIG. 10A. It has a height of Hb. FIG. 10B has a height that is ΔH more than for the prior art biopsy needle. A typical prior art biopsy needle used can be 0.040 inches in diameter at the portion proximal from the notch. The prior art example has a notch that is produced by cutting away about 0.025 inches of the needle's full diameter. This means that a typical height, Ha, for the prior art example of a notch 418 is about 0.015 inches. The cradle view illustrated in FIG. 10B can be fabricated by making Hb around 0.018 inches and then removing additional material near the center of the top surface. Heights Hb of more or less than 0.018 can be likewise produced.

If the areas shown in FIGS. 10A and 10B are the same and the material (typically stainless steel) is the same, it can be appreciated based on the above discussions that making Hb larger or smaller will result in a larger or smaller moment of inertia. Improved bending performance over the example of a prior art biopsy needle could also be achieved by simply increasing the thickness of keel 205 (FIG. 5A). This could provide an even larger increase in the moment of inertia over the prior art example; however, it would come at the expense of reducing the space for a tissue sample between cradle 604 and the inside wall of cannula 502. For core biopsy needles, it is important collect as large of a sample as possible for the given gauge of outer cannula used. So, such increase in the cross-sectional area of keel 205 is undesirable because it provides less area to contain a tissue sample. Increasing the moment of inertia of the needle without increasing the keel's cross-sectional area provides an important benefit of increasing the bend resistance of the needle without sacrificing tissue collection area.

FIG. 11 is an enlarged perspective view of an axial length of cradle 604 discussed above. As discussed, teeth 208 and 210 help reduce bending by improving the load constraint environment and keel 605 further helps reduce bending by improving the base strength of the cradle. When combined, both new features provide an important improvement in biopsy deflection performance.

While the underside of keel 605 has been illustrated as a continuous arc in cross-section, it need not be. For example, the underside of keel 605 can comprise one or more axially running ribs, or channels or can be scalloped. Similarly, top surface 612 need not be flat. It can be curved in cross-section or can similarly include axially running features such as ribs or grooves or can be roughened to help friction with a tissue sample over it.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A biopsy needle set comprising:
an axially extending outer cannula that has an inside wall and a central axis;
an axially extending core collector that slidingly fits inside the outer cannula and has a sharp distal tip and an axially extending cradle proximal from the tip;
wherein:
the cradle comprises a trough formed between two axially extending rows of teeth rising from lateral ends of an axially extending keel;
the cradle has an outer surface conforming to the cannula's inside wall;
the keel has a cross-section that decreases in thickness toward the lateral ends thereof;
the teeth of each row are axially spaced from each other; and
the outer surface of the cradle is sized to bear against the cannula's inside wall and thereby resist bending of the cradle due to forces acting on the tip and cradle while and when a portion of the cradle is extending distally from the cannula.

2. The biopsy needle set of claim 1, in which some cross-sections of the cradle intersect only said keel and are at axially spaced portions of the cradle that lack said teeth.

3. The biopsy needle set of claim 1, in which some cross-sections of the cradle intersect only one of said teeth.

4. The biopsy needle set of claim 1, in which some cross-sections of the cradle intersect two of said teeth, one from each of said rows.

5. The biopsy needle set of claim 1, in which some cross-sections of the cradle intersect at least one of said teeth and some intersect none or said teeth.

6. The biopsy needle set of claim 1, in which said keel is sector-shaped in cross-section.

7. The biopsy needle set of claim 1, in which the outer surface of the cradle extends over an arc of approximately half a circle.

8. The biopsy needle set of claim 1, in which the cannula has a distal tip beveled along a first angle and the tip of the core collector is beveled along a second angle opposite the first angle.

9. The biopsy needle set of claim 1, in which at least some of the teeth have sharpened tips.

10. The biopsy needle set of claim 1, in which the surface of said keel facing the cannula's inside wall is a continuous curve in cross-section.

11. The biopsy needle set of claim 1, in which the surface of said keel facing said though is flat.

12. The biopsy needle set of claim 1, in which the surface of said bearing against the inside wall of the cannula is continuous in cross-section.

13. A method of extracting a tissue sample comprising:
providing a cannula that has an inner wall and a core that slidingly fits inside the cannula;
wherein said core collector comprises a sharp distal tip and a cradle that is proximal from the tip and comprises an axially extending keel and two rows of teeth rising from lateral ends of the keel to thereby form a trough-shaped space between the keel and the teeth;
forcing the core collector distally out of the cannula and into tissue from an initial position in which the cradle in inside the cannula such that the outer surface of the cradle bears against the cannula's inside wall as the cradle is moving out of the cannula and thereby resists bending of the cradle portion that has exited the cannula due to tissue exerting bending forces on the tip and cradle; and
thereafter, forcing the cannula distally over the cradle to sever a sample of tissue that is between said trough shaped space and the cannula's inside wall.

14. The method of claim 13, in which the providing step comprises providing a core collector with a cradle some cross-sections of which intersect only said keel and are at axially spaced portions of the cradle that lack said teeth.

15. The method of claim 13, in which the providing step comprises providing a core collector with a cradle some cross-sections of which intersect only one of said teeth.

16. The method of claim 13, in which the providing step comprises providing a core collector with a cradle some cross-sections of which intersect two of said teeth, one from each of said rows.

17. The method of claim 13, in which the providing step comprises providing a core collector with a cradle some cross-sections of which intersect at least one of said teeth and some intersect none or said teeth.

18. The method of claim 13, in which the providing step comprises providing a core collector in which said keel is sector-shaped in cross-section.

19. The method of claim 13, in which the providing step comprises providing a core collector with a cradle the outer surface of which extends over an arc of approximately half a circle.

20. The biopsy needle set of claim 13, in which the providing step comprises proving a core collector in which at least some of said teeth have sharpened tips.
